(19) United States
(12) Patent Application Publication
(10) Pub. No.: US 2002/0035346 A1
(43) Pub. Date: Mar. 21, 2002

(54) Drug release (delivery system)

(76) Inventors: John R. Reynolds, Gainesville, FL (US); Hiep Ly, Webster, NY (US); Patrick John Kinlen, Fenton, MO (US); Vinod P. Menon, Woodbury, MN (US)

Correspondence Address: THOMPSON COBURN, LLP, ONE FIRSTAR PLAZA, SUITE 3500, ST LOUIS, MO, 63101, US

(21) Appl. No.: 09/929,590

(22) Filed: Aug. 14, 2001

Related U.S. Application Data
(60) Provisional application No. 60/225,082, filed on Aug. 14, 2000.

(51) Int. Cl. A61N 1/30; A61L 15/16

(52) U.S. Cl. 604/20; 424/447

(57) ABSTRACT

An electrically addressable release pad for use in a burst electrode drug release (delivery) system. The pad useable in this system comprises a polymer, preferably polypyrrole, which surrounds medicine. Once a section of the polymer receives an electrochemical potential sent from an electrode contained within the pad, a defined dose of medicine contained therein is released. The pad is designed to be worn by a patient, so that when the medication is released, its administered to the patient, without the patient having to do anything. The electrode typically can be a burst electrode, one with a unique electrical signature. The electrode can be triggered from a remote source.

This invention relates to a drug delivery system which comprises a pad with multiple pads loaded with electrically releasable drugs. More particularly, this invention relates to a pad comprising a polymeric zone having one or more dopant(s) or pharmaceutical drug(s) therein, releasable upon application of an electrical potential to the polymeric zone.

### BACKGROUND OF THE INVENTION

A major concern in the field of medicine is whether or not patients are taking their prescribed medicine. For the most part, medical personnel rely on the patients themselves to take prescribed medicines according to instructions given by the doctor or a pharmacist. Certain medications need to be taken at the same time every day, or at particular intervals within a day (e.g., every 4 hours). Everyday occurrences lead to people taking their medication at inappropriate times, thereby not taking prescribed medication to its utmost value. In order to work properly, certain medications require that the entire quantity of prescribed medication be taken (e.g., antibiotics and hormones). A problem with these types of medication is that some people stop taking medication when they stop feeling the symptoms for which they are taking medication.

### OBJECTS OF THE INVENTION

It is an object of this invention to provide a controlled drug delivery system, which releases a drug upon receipt of an electronic signal from a remote source.

It is an object of this invention to provide an improved process for medicating patients remotely by a health care system or doctor's office.

It is an object of this invention to provide an invention which can medicate a patient with medicinally effective doses of medicine at appropriate times.

It is also an object of this invention to create a remote medicating system which medicates patients upon instruction.

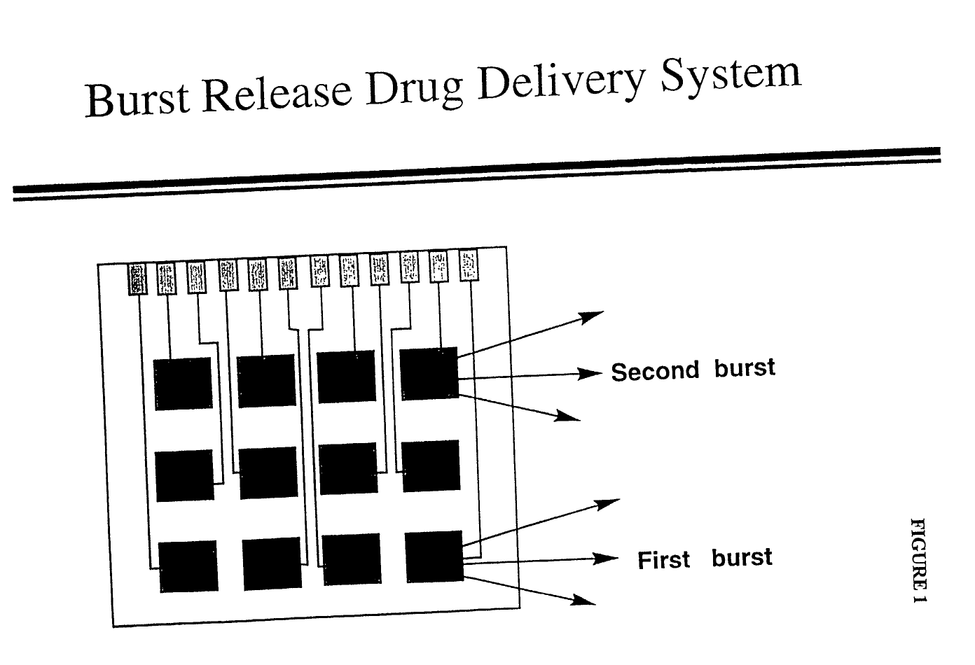

Another object of this system is to provide release pads, with one possible embodiment shown in FIG. 1, which can medicate patients which are flexible and bendable as well as durable and strong, such that physical contact with a patient will not cause a spontaneous medication or interfere with the proper doses needed to be given to patients.

It is another object of this invention to create a system of administering drugs that is safe to dispose, even unused portions, minimizing any chance of a third party (without a prescription) of administering the medication to himself.

These and other objects of the invention are further elucidated in more detail in the description of the invention which follows.

### BRIEF SUMMARY OF THE INVENTION

This invention comprises a drug release (delivery) system comprising an independent electrically addressable conductive pad or multiple pads, said pad(s) comprising an electroactive polymer containing a drug releasable therefrom upon application of a potential to the polymer whereby the application of an electrical potential or current to said pad(s) is communicated to said polymer whereupon said drug is effectively released or delivered from said polymer to a patient.

### DETAILED DESCRIPTION OF THE INVENTION

The invention herein is a cure for these aforementioned and other concerns. The present invention provides for a drug electrically release (delivery) system which utilizes an electroactive polymer preferably contained within a set of addressable release pads. In one embodiment these pads contain a prescribed medicine(s) which is medicated to a patient upon the release of an electrochemical potential from a unique type of electroactive polymer which can receive its signal from a source remote from the patient. Hence, the patient can be medicated remotely and/or automatically, without any more action than is required to be in contact with the release (delivery) pad.

An additional problem with medicating patients, is the waste of unused medicine. With this invention, there is limited medicinal waste. All or potentially all of the medicine contained in the pad will be prescribed when a signal is sent. A common problem, especially seen with children taking antibiotics, is that pills are prescribed to be taken for a long period of time (10 days or so), but the child stops taking the medication as soon as they feel well (usually within the first couple of days). This pad reduces this risk remarkably, because all that is necessary to receive the medical dose is a signal from the doctor's office, not an exertion for the patient.

Polymers useful as electroactive polymers in the pads of the instant invention comprise polymers with redox activity that can entrap and release ionic (both cationic and anionic) drugs. Conjugated polymers in general, such as the polyheterocycles and specifically polypyrrole and its derivatives, work quite well in these systems. Polypyrrole is a preferred polymer of the instant invention due to its ease of synthesis, oxidative stability, and benign nature.

Polymers useful in the pad release (delivery) system of the instant invention have been chosen by their electrochemical (redox) activity. Once an electrical potential interacts with these polymers, the polymers release a preset amount of a medicine(s), which was previously contained within the pad.

In operation of this invention, an electrical potential sent to the electroactive polymer triggers a release either a Faradaic nature (a gradual release linear with current or charge passed over an extended time period) or a unique burst release (in which a large release is achieved compared to an extremely small electronic impulse received). Depending on the type of electrode used, a patient can be medicated either with a large dose via a burst electrode type of release pad (as illustrated in FIG. 1), or can be medicated over a gradual time period through the use of a faradic electrode type of release pad.

Further in the operation of this invention, the pad optionally has a contact point, posted in said pad and conductive thereto, which can receive a signal from a remote signaling or instruction station. Nonlimiting examples of signaling stations include hospitals, doctors' offices or other similar health care facilities, where a doctor can ‘prescribe’ any medication contained within the pad to be administered to the patient in doses and at time intervals that the doctor finds necessary or desirable.

Remote computer programs could also utilized to prescribe the medication at set time intervals. Medication could then be delivered to patients without any over patient actions. If desired, a computer program could send the signal at a preset time, thereby medicating the patient when needed. This is especially important for antibiotics, steroids, hormones, or other similar drugs which require doses not just on a daily basis, but rather at a more specific time with a higher precision.

A set of pads with a telemetry system incorporated therein could be monitored by a computer program. Upon the occurrence of certain preset conditions, the computer program could dose the patient as necessary. This is especially useful for asthmatic patients, patients with high blood pressure, diabetics, or other patients with medical conditions of a similar nature. With this invention it would be possible for the computer program to recognize certain symptoms or problems arising within the patient's body before it is even noticeable by the patient. The computer program could then adjust the dose of medication to the proper level, or even alert a doctor to problems, so that the doctor could ‘prescribe’ a drug already contained within the pad to correct any problems before the patient feels any discomfort or symptoms of an oncoming illness or attack.

This invention also simplifies medicating non-human patients such as animals. Usually, getting an animal to take medication (especially if its a pet) is an exceedingly trying act (physically for the animal, and emotionally for the owners). Strategies invoked to medicate animals can vary the dose remarkably or could allow the medicines to be taken at inappropriate times. Acts such as combining medicine with food or chopping up pills into smaller pieces may improve the chance that an animal will obtain a medicinal dose, but there is no guarantee that the animal will eat all the food containing the medicine or that the medicine will be taken at appropriate times. Also, these acts (mixing medicine with food or chopping medicine into smaller pieces) could interfere with the coatings of such medications which usually are necessary components of the medicine to target or time release ingredients at different places in the body (so as to avoid a general release of medicine in the stomach). The pad system can easily be adapted to medicate animals. Dogs, cats, or the like could easily be medicated by a pad which could be contained within a collar of such animals. Other larger animals could easily be medicated by affixing the contacting pad onto the animal in a convenient manner.

The release pads of this invention are safely disposable. The unused drugs contained therein can not be released, except upon receipt of the proper signal. Without that signal, the drug remains contained within the release pad.

The electrodes herein contain an electroactive polymer with a biologically active ingredient incorporated thereon. Through an electrical potential or current interacting with the polymer, a release of the biologically active ingredient is achieved which delivers the biologically active ingredient to a patient wearing the pad or in contact with the pad. This release, depending on the polymer used, can be Faradaic in nature (a gradual release linear with current or charge passed over an extended time period), or can be burst in nature (in which a large release is achieved compared to an extremely small electronic impulse received).

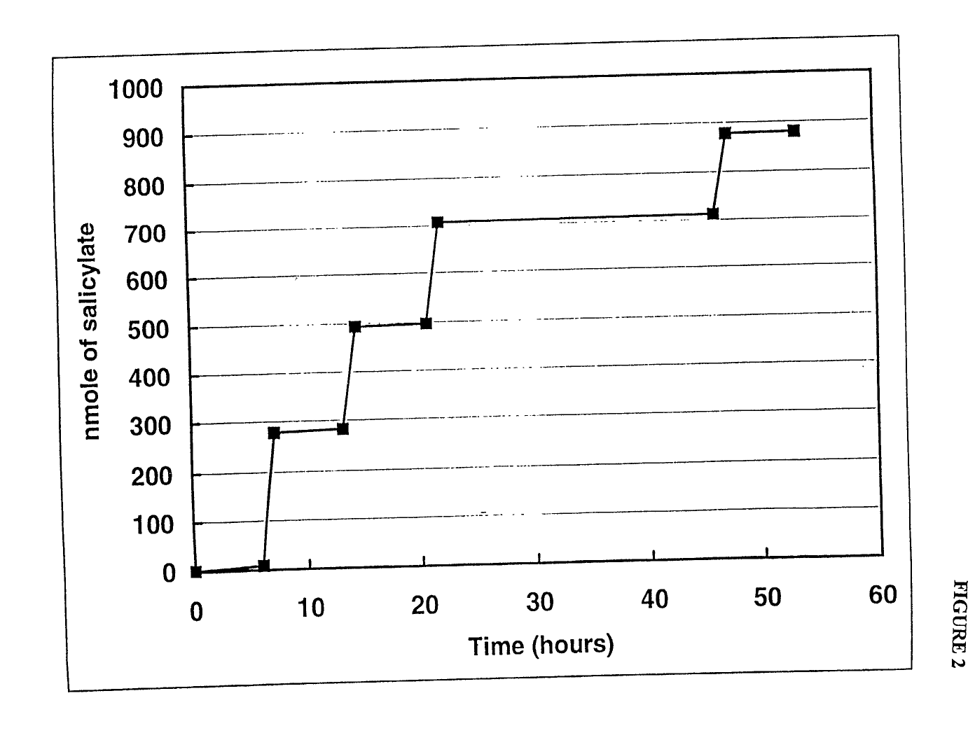

Conjugated and redox active polymers useful in this invention are chosen due to their electroactive qualities. Such polymers include, but are not limited to polyheterocycles and specifically polypyrroles and its derivatives. Through the use of polypyrrole, a large number of anionic molecules can be incorporated directly as charge compensating dopants. The use of polypyrroles allow for a large amount of an anionic biologically active ingredient to be incorporated thereon, or can allow a dopant species with polyanionic activity to be incorporated thereon, thereby allowing a cationic drug species to be infused therein. In order to prevent spontaneous release of the drugs, an overlayer should also be applied, preferably a hydrophobic polymer such as nafion, poly(vinyl acetate), poly(vinyl butyral) or the like. The use of an overlayer yields a stable electrode which can emit the drugs as desired (as shown in FIG. 2).

The electrical potential or current to trigger the drug release could be sent to the release pad from a remote instrument signal-generating source.

Pads created of such a nature have a huge potential to impact many different areas within the medical field. Home-patient care and emergency medicine find uses in that doctors can prescribe medicines when necessary, even if the doctor is quite a distance from the patient. The release pads also find use in veterinary medicine where its difficult even for expert veterinarians to ensure their patients are taking their required medicine at the proper times. Uses for these pads are also as far reaching as providing medicine for astronauts or people with potential illnesses in very remote locations where meeting with a doctor in person is not possible. Uses for this pad can also be found in the daily life of asthmatics or patients with heart conditions who simply have too many different types of medicine to keep track of.

The remote pads benefit from their physical flexibility and bendability. Pressures or forces acting upon the pad will not cause a spontaneous release of medicine or cause the release pads to fail in delivery of the proper doses required to their wearers due to damage.

Drugs useful in the pads of the instant invention include NSAIDS, analgesics, antihistamine, antitussives, decongestants, expectorants, steroids, enzymes, proteins, antibiotics, hormones, and mixtures thereof and the like.

Specific types of biologically active ingredients useful in the pads of the instant invention include nutritional supplements, anti-inflammatory agents (e.g. NSAIDS such as s-ibuprofen, ketoprofen, fenoprofen, indomethacin, meclofentamate, mefenamic acid, naproxen, phenylbutazone, piroxicam, tolmetin, sulindac, and dimethyl sulfoxide), antipyretics, anesthetics including benzocaine, pramoxine, dibucaine, diclonine, lidocaine, mepiracaine, prilocaine, and tetracaine; demulcents; analgesics including opiate analgesics, non-opiate analgesics, non-narcotic analgesics including acetaminophen and astringent including calamine, zinc oxide, tannic acid, Hamamelis water, zinc sulfate; natural or synthetic steroids including triamcinolone, acetonide, perdnisone, beclomethasone dipropionate; asthmatic drugs including terbutaline sulfate, albuterol, leukotriene receptor antagonists; electrolytes, metals and minerals; antianxiety and antidepressant agents; antimicrobial and antiviral agents; antihistamines; immune-suppression agents; cholesterol-lowering agents; cardiac and high-blood pressure agents and mixtures thereof.

The examples herein are only illustrations of various embodiments of this invention and are not intended to limit it in anyway.

### EXAMPLE

A patterned burst release drug delivery system is prepared in accordance with this invention as depicted in FIG. 1. The release pads are electrochemically coated with polypyrrole/salicylate and then top coated with PVOH (polyvinyl alcohol) which is then crosslinked. All of the pads are individually addressable and electrochemically triggered. Total release of the drug occurs when an individual pad is stimulated, without triggering release from the remaining pads. Spontaneous release of the drug in the pads of the instant invention is not significant. Pulsatile release is also possible through the use of an array of burst release electrodes.

Thus, it is apparent that there has been provided, in accordance with the instant invention, a process that fully satisfies the objects and advantages set forth herein above. While the invention has been described with respect to various specific examples and embodiments thereof, it is understood that the invention is not limited thereto and many alternatives, modifications and variations will be apparent to those skilled in the art in light of the foregoing description. Accordingly, it is intended to embrace all such alternatives, modifications and variations as fall within the spirit and broad scope of the invention.

What is claimed is:

1. An electrode drug release (delivery) system comprising an electroactive polymer having at least one electrically addressable release pad(s), said system comprising:
a. at least one electrode system(s) containing a drug releasable therefrom upon application of a potential to said electrode which is in electrical communication with
b. at least one independently electrically addressable release pad(s),
whereby the application of an electrical potential or current to said pad(s) is communicated to said electrode(s) causing said drug to be effectively released or delivered.

2. The system of claim 1 wherein said electrode systems comprises more than one electrode wherein each electrode is independent of one another.

3. The system of claim 1 wherein more than one pad is utilized in said electrode drug release system.

4. The system of claim 3 wherein multiple electrodes and multiple pads are employed therein.

5. The system of claim 4 wherein said electrode(s) is an electroactive polymer.

6. The system of claim 5 wherein said electroactive polymer is polypyrrole.

7. The system of claim 6 wherein said drug is selected from the group consisting of NSAIDS, analgesics, antihistamine, antitussives, decongestants, expectorants, steroids, enzymes, proteins, antibiotics, hormones, and mixtures thereof and the like.

8. The system of claim 7 wherein said drug is selected from the group consisting of nutritional supplements, s-ibuprofen, ketoprofen, fenoprofen, indomethacin, meclofentamate, mefenamic acid, naproxen, phenylbutazone, piroxicam, tolmetin, sulindac, dimethyl sulfoxide, benzocaine, pramoxine, dibucaine, diclonine, lidocaine, mepiracaine, prilocaine, and tetracaine, opiate analgesics, non-opiate analgesics, non-narcotic analgesics, acetaminophen, calamine, zinc oxide, tannic acid, Hamamelis water, zinc sulfate, triamcinolone, acetonide, prednisone, beclomethasone dipropionate, terbutaline sulfate, albuterol, leukotriene receptor antagonists, electrolytes, metals, minerals, antianxiety, antidepressant agents, antimicrobial agents, antiviral agents, antihistamines, immune-suppression agents, cholesterol-lowering agents, cardiac, high-blood pressure agents, mixtures thereof and the like.

9. A method of providing medicine to a patient comprising:
a) placing a burst electrode drug release (delivery) system in external contact with a patient, said system comprising at least one electrically addressable release pad(s) and at least one burst electrode system(s) containing a drug releasable therefrom upon application of a potential to said electrode, said electrode in electrical communication with said release pad(s), and
b) applying an electrical potential or current to said release pad(s), wherein the potential or current is communicated to said burst electrodes whereupon said drug is effectively released or delivered to the patient.

10. The method of claim 9 wherein the electrode is an electroactive polymer.

11. The method of claim 9 wherein said drug is selected from the group consisting of NSAIDS, analgesics, antihistamine, antitussives, decongestants, expectorants, steroids, enzymes, proteins, antibiotics, hormones, mixtures thereof and the like.

12. The method of claim 9 wherein said drug is selected from the group consisting of nutritional supplements, s-ibuprofen, ketoprofen, fenoprofen, indomethacin, meclofentamate, mefenamic acid, naproxen, phenylbutazone, piroxicam, tolmetin, sulindac, dimethyl sulfoxide, benzocaine, pramoxine, dibucaine, diclonine, lidocaine, mepiracaine, prilocaine, and tetracaine, opiate analgesics, non-opiate analgesics, non-narcotic analgesics, acetaminophen, calamine, zinc oxide, tannic acid, Hamamelis water, zinc sulfate, triamcinolone, acetonide, prednisone, beclomethasone dipropionate, terbutaline sulfate, albuterol, leukotriene receptor antagonists, electrolytes, metals, minerals, antianxiety, antidepressant agents, antimicrobial agents, antiviral agents, antihistamines, immune-suppression agents, cholesterol-lowering agents, cardiac, high-blood pressure agents, mixtures thereof and the like.

13. The method of claim 9 wherein said drug is prescribed from a remote location from the patient, by an electrical signal sent to said release pad, said electrical sent causing a release of said drug to the patient.

14. A medical application system which comprises a burst electrode drug release (delivery) system comprising an electroactive polymer having at least one electrically addressable release pad(s), said system comprising:
a. at least one burst electrode system(s) containing a drug releasable therefrom upon application of a potential to said electrode which is in electrical communication with
b. at least one independently electrically addressable release pad(s),
whereby the application of an electrical potential or current to said pad(s) is communicated to said burst electrode system(s), whereupon said drug is effectively released or delivered.

15. The system of claim 14 wherein said electrode system(s) comprises more than one electrode independent from one another.

16. The system of claim 14 wherein more than one pad is utilized in said electrode drug release system.

17. The system of claims 15 or 16 wherein multiple electrodes and multiple pads are employed therein.

18. The system of claim 17 wherein said drug is selected from the group consisting of NSAIDS, analgesics, antihistamine, antitussives, decongestants, expectorants, steroids, enzymes, proteins, antibiotics, hormones, and mixtures thereof and the like.

19. The system of claim 18 wherein said drug is selected from the group consisting of nutritional supplements, s-ibuprofen, ketoprofen, fenoprofen, indomethacin, meclofentamate, mefenamic acid, naproxen, phenylbutazone, piroxicam, tolmetin, sulindac, dimethyl sulfoxide, benzocaine, pramoxine, dibucaine, diclonine, lidocaine, mepiracaine, prilocaine, and tetracaine, opiate analgesics, non-opiate analgesics, non-narcotic analgesics, acetaminophen, calamine, zinc oxide, tannic acid, Hamamelis water, zinc sulfate, triamcinolone, acetonide, prednisone, beclomethasone dipropionate, terbutaline sulfate, albuterol, leukotriene receptor antagonists, electrolytes, metals, minerals, antianxiety, antidepressant agents, antimicrobial agents, antiviral agents, antihistamines, immune-suppression agents, cholesterol-lowering agents, cardiac, high-blood pressure agents, mixtures thereof and the like.

20. A method of administering medication to a physically remote patient through a burst electrode drug release (delivery) system, said system comprising an electroactive polymer having at least one electrically addressable release pad(s), said system further comprising:
a. at least one burst electrode system(s) containing a drug releasable therefrom upon application of a potential to said electrode which is in electrical communication with
b. at least one independently electrically addressable release pad(s),
wherein said method comprises the application of an electrical potential or current to said pad(s) which is then communicated to said burst electrodes whereupon said drug is effectively released or delivered to said patient.

21. The method of claim 20 wherein said patient is a human patient.

22. The method of claim 20 wherein said patient is an animal patient.

23. The method of claim 20 wherein telemetry is employed to transfer biomedical information from said patient to said doctor.

24. The method of claim 23 wherein said doctor employs electrical communication means to transmit an activating signal to said medical application system whereby medicine is made effectively available to said patient.

25. An article of manufacture which comprises an electronically conductive polymer which surrounds a medication, an electrode which upon the receipt of a signal releases an electrochemical potential which interacts with said conductive polymer, causing the release of said medication.

26. The article of manufacture of claim 25 wherein the electrode is a burst electrode.

27. The article of manufacture of claim 25 and 26 wherein the polymer is polypyrrole.

28. The article of manufacture of claims 25 and 27 wherein the article of manufacture is an addressable release pad and is in contact with a patient which receives said dose of medication.

29. A process of administering medicine to a patient through a burst electrode drug release system comprising sending a signal through a signaling means to a pad in contact with a patient, said pad comprising at least one electrode to receive said signal, wherein said electrode upon receipt of the signal causes the release of medicine to medicate the patient.

30. The process of claim 29 wherein the receipt of the signal by the electrode causes the patient to be medicated by the release of an electrical potential.

31. The process according to claim 30 wherein the electrical potential interacts with electroactive polymers, said electroactive polymers containing said medicine.

32. The process according to claim 29 wherein the electrode is faradic.

33. The process according to claim 29 wherein the electrode is a burst release electrode.

34. The process of claims 32 and 33 wherein the electrode is an electroactive polymer.

35. The process of claim 34 wherein the electroactive polymer is polypyrrole.

36. The method of claim 29 wherein the patient is a human being.

37. The method of claim 29 wherein the patient is an animal.

38. The process according to claim 29 wherein said medicine comprises the group of NSAIDS, analgesics, antihistamine, antitussive, decongestant, expectorants, steroids, enzymes, proteins, antibiotics, hormones, mixtures thereof and the like.

39. An article of manufacture through which a doctor, remote from a patient, can by telemetry contained within the article of manufacture, observe a patient's vital signs and after observation of the patients vital signs, can through a signaling device, send to said article of manufacture an electronic signal which causes the release of a selected medicinal compound.

40. The article of manufacture of claim 39 wherein more than one medicinal compound is contained within the article of manufacture.

41. The article of manufacture of claim 39 wherein a separate signal can be sent to release each different medicine contained therein.

42. The article of manufacture of claim 39 and 41 wherein the article of manufacture is specifically fitted for contact with animals.

43. The article of manufacture of claim 42, wherein the article of manufacture is an animal collar.

44. A controlled drug delivery system comprising at least one electrically addressable release pad, wherein the application of an electrical potential or current to said pad communicates to a burst electrode contained within said controlled drug delivery system, causes the release of a biologically active ingredient.

45 The system of claim 44 further comprising an electroactive polymer with said biologically active ingredient incorporated thereon.

46. The system of claim 45 wherein a second polymer is applied as an overlayer to the electroactive polymer and said biologically active ingredient incorporated thereon.

47. The system of claim 45 wherein the biologically active ingredient is anionic.

48. The system of claim 46 wherein a polyanionic dopant is incorporated into the electroactive polymer and the biologically active ingredient is cationic.

49. The system of claims 47 and 48 wherein the biologically active ingredient is released in a burst release.

50. The system of claims 47 and 48 wherein the biologically active ingredient is released in a Faradaic manner.

51. The system of claims 49 and 50 wherein the biologically active ingredient is a pharmaceutical compound.

52. The system of claim 51 wherein the electric potential or current is triggered by a signal from a remote source.

53. The system of claim 52 wherein the signal can be modulated to correspond to the release of more than one type of biologically active ingredient.
